(19) United States
(12) Patent Application Publication
(10) Pub. No.: US 2023/0372259 A1
(43) Pub. Date: Nov. 23, 2023

(54) POLYMER NANOPARTICLE, METHOD FOR THE MANUFACTURE THEREOF, AND METHOD FOR INTRACELLULAR DELIVERY OF A CARGO

(71) Applicant: UNIVERSITY OF MASSACHUSETTS, Boston, MA (US)

(72) Inventor: Sankaran Thayumanavan, Amherst, MA (US)

(21) Appl. No.: 18/197,957

(22) Filed: May 16, 2023

Related U.S. Application Data
(60) Provisional application No. 63/343,655, filed on May 19, 2022.

(51) Int. Cl. A61K 9/51; C08L 89/00; A61K 38/46; A61K 9/50; C12N 15/113

(52) U.S. Cl. CPC A61K 9/5153; C08L 89/00; A61K 38/465; A61K 9/5192; A61K 9/5015; C12N 15/113; C12N 2310/14

(57) ABSTRACT

A polymer nanoparticle includes a crosslinked polymer complex including a cargo encapsulated in a crosslinked polymer network; and a coating on the crosslinked polymer complex. The coating is derived from a cellular membrane. The polymer nanoparticles described herein can advantageously be used to deliver a cargo molecule (e.g., a nucleic acid) to a target location, for example upon administration to a subject in need of therapy.

### CROSS REFERENCE TO RELATED APPLICATION

This application claims priority to U.S. Provisional Application No. 63/343,655, filed May 19, 2022, the contents of which is hereby incorporated by reference in its entirety.

### FEDERAL RESEARCH STATEMENT

This invention was made with government support under award number GM-136395 awarded by the National Institute of General Medical Sciences/National Institutes of Health. The government has certain rights in the invention.

### BACKGROUND

Self-assembly that relies on non-covalent intermolecular interactions, comprising single or multi-component molecular building blocks, plays a fundamentally important role in many biological processes and in the development of novel functional materials. See, e.g., Whitesides, et al. 1991 Science, 254 (5036), 1312-9; Zhang, 2003. Nat. Biotechnol. 21 (10), 1171-1178. However, designing and assembling multiple molecular entities to generate a predictable and controlled supramolecular assembly have considerable challenges; but if achieved, this can propel the design of materials with functional capabilities that are currently not attainable.

Poor cellular internalization, serum instability, rapid clearance, severe cytotoxicity and potential immunological flare-ups have been identified as the critical barriers for such promising technology. Potential solutions like chemically modified siRNAs and viral vectors have yet to overcome issues owing to cytotoxicity, stability, immunogenicity and reduced silencing ability upon structural modifications. See, e.g., Nguyen, et al. 2012 Acc. Chem. Res. 45 (7), 1153-1162; Wang, et al. 2010 Aaps J. 12 (4), 492-503; Gallas, et al. 2013 Chem. Soc. Rev. 42 (20), 7983-7997; Majumder, et al. 2018 Chem. Commun. 54 (12), 1489-1492; Roy, et al. 2009 Biomacromolecules 10 (8), 2189-93; Zheng, et al. 2012 ACS Nano 6 (11), 9447-54; Xue, et al. 2014 Nanomedicine (London, U.K.) 9 (2), 295-312; Wang, et al. 2016 Chem. Commun. 52 (6), 1194-1197.

Accordingly, there remains a continuing need in the art for improved delivery systems capable over delivering various therapeutic agents, including for example, hydrophobic therapeutic agents and large hydrophilic bio-macromolecules such as proteins and nucleic acids. It would be particularly advantageous to provide a delivery system capable of providing enhanced cellular uptake and high endosomal escape of the therapeutic agent.

### SUMMARY

A polymer nanoparticle comprises a crosslinked polymer complex comprising a cargo encapsulated in a crosslinked polymer network; and a coating on the crosslinked polymer complex, wherein the coating is derived from a cellular membrane.

Another aspect is a method of making the polymer nanoparticle comprising: contacting an amphiphilic polymer and a cargo molecule to form a polymer complex; crosslinking the polymer to form a crosslinked polymer network entrapping the cargo molecule therein; and contacting the crosslinked polymer complex with a cell under conditions effective to provide the coating derived from a cellular membrane.

Another aspect is a method for delivering a cargo, the method comprising: administering the polymer nanoparticle to a subject in need of therapy.

The above described and other features are exemplified by the following figures and detailed description.

### BRIEF DESCRIPTION OF THE DRAWINGS

The following figures represent exemplary embodiments.

FIG. 1 is a schematic illustration of preparation of a polymer nanoparticle according to an aspect of the present disclosure.

FIG. 2 is a schematic illustration of preparation of a polymer nanoparticle according to an aspect of the present disclosure.

FIG. 3 shows average diameter and zeta potential for polymer nanoparticles of the present examples.

FIG. 4 shows transmission electron microscope images for polymer nanoparticles of the present examples.

FIG. 5 shows results of flow cytometry of polymer nanoparticles according to an aspect of the present disclosure in three cell lines and at varying polymer:cell membrane weight ratios.

FIG. 6 shows confocal microscope images of polymer nanoparticles of the present examples after incubation with MDA-MB-231 cells.

FIG. 7 shows confocal microscope images of polymer nanoparticles indicating the uptake mechanism.

FIG. 8 is a schematic illustration of preparation of a polymer nanoparticle according to an aspect of the present disclosure.

FIG. 9 shows flow cytometry results after incubation of polymer nanoparticles of the examples after incubation with MDA-MB-231 cells.

FIG. 10 shows flow cytometry results after incubation of polymer nanoparticles of the examples after incubation with HEP-3B cells.

### DETAILED DESCRIPTION

The present inventors have discovered a virus-inspired nanoparticle delivery platform for the intracellular delivery of cargo such as nucleic acids, proteins, antibodies, and hydrophobic molecules to cells. The nanoparticles of the present disclosure can be assembled in simple steps, with the ability to tune various factors of macromolecular delivery, including biological cargo encapsulation, cytosolic delivery, and cellular targeting. This level of tunability offers the unique opportunity to provide new formulations for intracellular delivery of nucleic acids, proteins, antibodies, hydrophobic molecules, and combinations thereof.

The macromolecular delivery challenge is defined by four distinct, but interconnected, processes: (1) encapsulation of the macromolecular cargos in the formulation; (2) efficient cellular/tissue targeting; (3) efficient cellular entry and localization to the specific sub-cellular compartment; and (4) release of cargos in their functional form in that compartment for specific target engagement. See, e.g., Qin, X. et al., Adv. Mater. 2019, 31 (46); Dutta, K. et al., Adv. Funct. Mater. 2021, 31 (24). The bio-inspired molecular design and the formulation protocols described in the present disclosure uniquely offer to address the grand challenge of intracellular macromolecular delivery, as each of these processes can be independently tuned by varying different components of the formulation.

In nature, a virus is able to use electrostatic pressure to efficiently inject nucleic acids into the cytosol. The electrostatic pressure in the viral capsid is due to the high density of negative charge that is present in the encapsulated RNA molecule without charge compensation. See, e.g., Brandariz-Nuñez, A. et al. Elife 2019, 8; Bauer, D. W. et al. J. Am. Chem. Soc. 2013, 135 (30), 11216-11221. To create such a system in a polymeric nanoparticle, a polymer nanoparticle that encapsulates a cargo such as nucleic acids without charge compensation has been employed, thus creating a system that is under significant electrostatic pressure. See, e.g., Dutta, K. et al. ACS Appl. Mater. Interfaces 2019, 11 (28), 24971-24983. In this process, the polymer alone is used for encapsulation and thus controls the encapsulation of the cargo during the formulation and their release at a specific intracellular location. A cell membrane coating dictates the surface properties of the nanoparticle, which controls its interaction with cells and sub-cellular compartments and can offer targeting to specific cells and tissues. Once they reach the target location, these nanoparticles can be programmed to release their contents because of the presence of specifically triggerable crosslinkers in the nanoparticles. The ability to breakdown the parts of the macromolecular delivery challenge and address them independently with structural components that are seamlessly integrated into a simple formulation scheme highlights the innovation of the present approach to tackling this grand challenge.

Accordingly, an aspect of the present disclosure is a polymer nanoparticle. The polymer nanoparticle comprises a crosslinked polymer complex and a coating on the crosslinked polymer complex. The polymer nanoparticles can have an average cross-sectional diameter of, for example, 5 to 300 nanometers. The size of the polymer nanoparticles can be characterized, for example, by dynamic light scattering (DLS) or transmission electron microscopy (TEM).

The crosslinked polymer complex comprises a cargo encapsulated in a crosslinked polymer network. The crosslinked polymer complex of the present disclosure is advantageous because i) the high binding affinity results in efficient capture of a cargo (e.g., siRNAs) inside the assemblies; (ii) although electrostatics is utilized to capture the cargo, the residual assembly is non-cationic due to an in situ crosslinking protocol that removes the cationic charge on the polymer, yet incarcerates the cargo; (iii) the surface charge of the assemblies is non-cationic; and (iv) the cargo can be released using a trigger that corresponds to the operational environment of the cargo.

In an aspect, the crosslinked polymer complex comprises a crosslinked copolymer. The crosslinked copolymer can comprise repeating units according to Formula (I) and Formula (II)

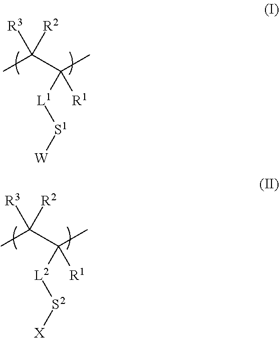

wherein in the foregoing Formulas, R¹ is independently at each occurrence hydrogen, a C₁₋₁₂ alkyl group, or a halogen; R² and R³ are independently at each occurrence hydrogen, a C₁₋₆ alkyl group, a C₁₋₁₆ alkyloxy group, or halogen; L¹ and L² are independently at each occurrence a linking group; S¹ and S² are independently at each occurrence a single bond or a spacer group; W is a hydrophobic group; and X is a group comprising a crosslinking moiety. In an aspect, the spacer group S¹ and S² can be a C₁₋₁₆ alkylene group. In an aspect, each occurrence of R² and R³ are hydrogen, and each occurrence of R¹ is a methyl group. In an aspect, L¹ and L² are independently at each occurrence an ester (—(C═O)O—) or an amide (—(C═O)NH—) linking group. In an aspect, each occurrence of L¹ and L² are ester (—(C═O)O—) linking groups. In an aspect, each occurrence of S¹ and S² is an ethylene group.

In an aspect, W in Formula (I) is a hydrophobic group, which can include, for example, hydrocarbons. Suitable hydrophobic groups lack the ability to hydrogen bond and their surface free energy is relatively low, resulting in hydrophobicity. In an aspect, W can comprise a C₁₋₃₀ alkyl group, a C₆₋₃₀ alkyl group, a C₉₋₃₀ alkyl group, a C₁₂₋₃₀ alkyl group, a C₁₅₋₃₀ alkyl group, a C₁₈₋₃₀ alkyl group, a C₆₋₂₄ alkyl group, a C₁₂₋₂₄ alkyl group, a C₁₅₋₂₄ alkyl group, a C₁₀₋₂₀ alkyl group, a C₉₋₁₅ alkyl group, or a C₁₂₋₁₅ alkyl group. The foregoing alkyl groups can be linear, branched, or cyclic alkyl groups.

In an aspect, X in Formula (II) comprises a crosslinking moiety and a cationic group. The term “crosslinking” as used herein refers to forming a bond that links one polymer chain to another polymer chain (intermolecular crosslinking) or that links one portion of a polymer chain to another portion of the same polymer chain (intramolecular crosslinking) or a combination thereof. The term “crosslinking moiety” as used herein refers to a chemical moiety that is either capable of forming a crosslink or is a chemical moiety that is crosslinked, either within the same polymer molecule or between different polymer molecules. The term “non-crosslinking group” refers to a chemical group unable to form a crosslink (e.g., a chemical crosslink).

In an aspect, X in Formula (II) can comprise a disulfide group. In an aspect, X in Formula (II) can comprise a quaternary ammonium cation. In an aspect, X in Formula (II) can comprise a sulfonium cation. In an aspect, X in Formula (II) can comprise a group of the Formula

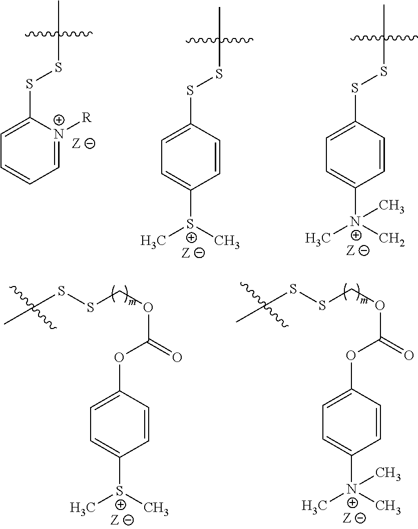

wherein R is a C₁₋₁₅ alkyl group, and Z is a counter ion. In an aspect, R can be a C₁₋₁₂ alkyl group, a C₁₋₆ alkyl group, a C₁₋₃ alkyl group, for example a methylene group (i.e., a C₁ group). Any suitable counter ion can be employed. In an aspect, the counter ion Z can comprise a halide, an acetate, a carbonate, a sulfate, a mesylate, a maleate, a citrate, and the like or a combination thereof.

In an aspect, the ratio of the repeating units according to Formula (I) and Formula (II) is in the range of 5:95 to 95:5. Within this range, the ratio of the repeating units according to Formula (I) and Formula (II) can be 10:90 to 90:10, or 20:80 to 80:20.

In an aspect, at least a portion of the repeating units according to Formula (II) are replaced with the repeating units according to Formula (IIA)

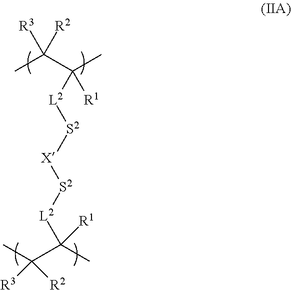

wherein R¹, R², R³, L², and S² are as defined above, and X′ represents a crosslinked group (e.g., a group resulting from the reaction of two crosslinkable groups X of Formula (II)). In an aspect, X′ can be a disulfide group.

It will be understood that the repeating units according to Formula (IIA) are derived from the repeating units according to Formula (II), and are a result of a crosslinking reaction between repeating units according to Formula (II) on a different polymer chain and/or at a different portion of the same polymer chain.

In an aspect, the crosslinked copolymer can further comprise repeating units according to Formula (III)

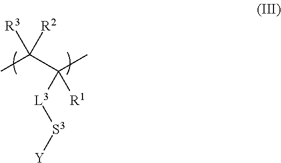

wherein R¹ is independently at each occurrence hydrogen, a C₁₋₁₂ alkyl group, or a halogen; R² and R³ are independently at each occurrence hydrogen, a C₁₋₆ alkyl group, a C₁₋₁₆ alkyloxy group, or halogen; L³ is a linking group; S³ is a single bond or a spacer group; and Y is a non-crosslinking group. In an aspect, the spacer group S³ can be a C₁₋₁₆ alkylene group. In an aspect, each occurrence of R² and R³ are hydrogen, and each occurrence of R¹ is a methyl group. In an aspect, L³ is independently at each occurrence an ester (—(C═O)O—) or an amide (—(C═O)NH—) linking group. In an aspect, L³ is an ester (—(C═O)O—) linking group. In an aspect, S³ is an ethylene group. In an aspect, Y can be a C₁₋₂₀ alkyl group, a C₁₋₁₅ alkyl group, a C₁₋₁₂ alkyl group, a C₁₋₉ alkyl group, a C₁₋₆ alkyl group, a C₁₋₃ alkyl group, a C₃₋₂₀ alkyl group, a C₆₋₂₀ alkyl group, a C₆₋₁₅ alkyl group, a C₉₋₂₀ alkyl group, a C₁₂₋₂₀ alkyl group, a C₃₋₁₅ alkyl group, a C₃₋₁₂ alkyl group, a C₃₋₆ alkyl group, or a C₆₋₁₂ alkyl group. The foregoing alkyl groups can be linear or branched, and can optionally be substituted, for example with an aromatic moiety.

In an aspect, the crosslinked copolymer can be a random copolymer or a block copolymer. In an aspect, the crosslinked polymer nanoparticle can comprise a random copolymer, a block copolymer, or a combination thereof.

In an aspect, the copolymer can have a number average molecular weight (Mₙ) of, for example, 1,000 to 200,000 grams per mole, for example 1,000 to 150,000 grams per mole, or 1,000 to 100,000 grams per mole, or 1,000 to 75,000 grams per mole, or 1,000 to 50,000 grams per mole, or 5,000 to 200,000 grams per mole, or 10,000 to 200,000 grams per mole, or 50,000 to 200,000 grams per mole, or 100,000 to 200,000 grams per mole. Molecular weight can be determined (prior to crosslinking) by gel permeation chromatography, for example eluting with tetrahydrofuran relative to polystyrene standards.

In an aspect, the crosslinked polymer network can comprise a copolymer according to Formula (IV)

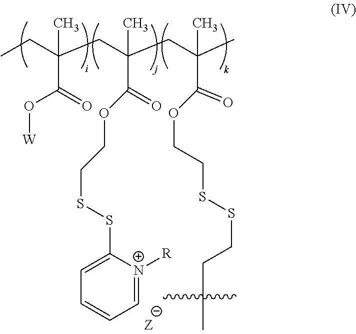

wherein W is as defined previously, R is a C₁₋₁₅ alkyl group, and Z is a counter ion. In an aspect, R can be a C₁₋₁₂ alkyl group, a C₁₋₆ alkyl group, a C₁₋₃ alkyl group, or a methylene group. The curved line in Formula (IV) indicates a point of attachment to another polymer chain and/or another portion of the same polymer chain. Stated another way, the repeating units indicated by “k” in the foregoing Formula represent a crosslinked repeat unit of the polymer structure.

A ratio of i:j can be, for example, 1:9 to 9:1, or 2:8 to 8:2, and 1 to 95 mole percent of j can be substituted by k. In an aspect, each of i and j can independently be 1 to 500, or 1 to 300, or 1 to 200, or 1 to 100, or 1 to 50, or 1 to 20, or 1 to 10, or 10 to 500, or 50 to 500, or 100 to 500, or 200 to 500, of 10 to 100, or 10 to 50, or 10 to 20, or 20 to 200, or 20 to 100.

In an aspect, k can be 0. Preferably, k can be greater than 0 to 500, or 1 to 500, or 0 to 475, or 1 to 475, or 1 to 300, or 1 to 200, or 1 to 100, or 1 to 50, or 1 to 20, or 1 to 10, or 10 to 500, or 50 to 500, or 100 to 500, or 200 to 500, or 10 to 100, or 10 to 50, or 10 to 20, or 20 to 200, or 20 to 100.

In an aspect, the crosslinked network can have a crosslink density of 1 to 80%, relative to the total number of structural units in the polymer. For example, the crosslinking density can be 10 to 60%, or 10 to 30%, or 30 to 60%, each relative to the total number of structural units in the polymer.

The crosslinked polymer complex further comprises a cargo entrapped in the crosslinked copolymer network. The term “entrapped” as used herein refers to the encapsulation, immobilization, or other capture of a cargo molecule by the crosslinked copolymer. Preferably, the cargo is physically trapped within the crosslinked copolymer and is not covalently bound to the crosslinked copolymer.

The cargo can comprise a hydrophobic molecule, a nucleic acid, a protein, an antibody, or a combination thereof. In an aspect, the cargo can comprise a nucleic acid, a protein, or a combination thereof.

In an aspect, the cargo can comprise a nucleic acid. The nucleic acid can comprise single-stranded or double-stranded RNA or DNA, or a derivative or analog thereof. For example, the nucleic acid can comprise dsRNA, siRNA, mRNA, ncRNA, microRNA, catalytic RNA, gRNA, sgRNA, DNAs, oligonucleotides, aptamers, genes, plasmids, or a derivative or analog thereof. In a specific aspect, the nucleic acid can comprise siRNA. In a specific aspect, the nucleic acid can comprise sgRNA.

In an aspect, the cargo can comprise a protein. As used herein, the term “protein” refers to a polypeptide, a polymer of amino acid residues, and is not limited to a minimum length. Thus, peptides, oligopeptides, dimers, multimers, enzymes, antibodies, aptamers, and the like are included within the definition. Both full-length proteins and fragments thereof are encompassed by the definition. The term can also include post-expression modifications of the polypeptide, for example glycosylation, acetylation, phosphorylation, and the like. Furthermore, a “protein” may refer to a polypeptide which includes modifications, such as deletions, additions, and substitutions to the native sequence provided that the protein maintains the desired activity. In an aspect, the protein can comprise a CRISPR-associated protein, for example Cas9.

In an aspect, the cargo can comprise a combination of a protein and a nucleic acid.

The cargo can be entrapped in the polymer network at a loading of 0.2 to 70 weight percent, based on the total weight of the crosslinked polymer complex. Within this range, the cargo can be present in an amount of 0.5 to 70 weight percent, or 2 to 70 weight percent, or 10 to 70 weight percent, or 0.2 to 30 weight percent, or 0.2 to 10 weight percent, or 0.2 to 5 weight percent, each based on the total weight of the crosslinked polymer complex.

In an aspect, the crosslinked polymer complex can be de-crosslinked partially or completely upon contact with a biological or chemical stimulus. For example, X in Formula (II) can comprise a pH-sensitive functional group, a redox-sensitive functional group, or a combination thereof. In an aspect, the crosslinked polymer network can be decrosslinked triggered by an intracellular reducing environment (e.g., an elevated glutathione concentration). Decrosslinking the polymer network can result in release of the nucleic acid from the polymer nanoparticle.

The crosslinked polymer complex can be prepared by contacting an amphiphilic polymer and a cargo molecule to form a polymer complex and crosslinking the polymer of the polymer complex to form a crosslinked polymer network entrapping the cargo therein. For example, the crosslinked polymer complex can be prepared according to methods described in U.S. Publication No. 2020/0332047, the contents of which are incorporated by reference herein in their entirety for all purposes.

The polymer nanoparticle of the present disclosure further comprises a coating on the crosslinked polymer complex. The coating is derived from a cellular membrane. In an aspect, the coating can be in the form of a lipid bilayer. In an aspect, the lipid bilayer, being derived from a natural cell membrane, can comprise biological lipids, including polar phospholipids, glycolipids, storage lipids, and lipid-associated sterols. The coating can further comprise lipid-associated biomolecules from native cellular membranes, such as membrane associated proteins and post-translationally modified proteins. In an aspect, the coating can be derived from membrane lipids which have been isolated from a native cellular membrane. The cell from which the coating is derived can be a human cell, an animal cell, or a plant cell. In an aspect, the coating can be derived from a blood cell, a tumor cell, a cancer cell, an immune cell, a stem cell, a neuronal cell, an epithelial cell, or an endothelial cell. In an aspect, the coating can be derived from a red blood cell, a mesenchymal stem cell, or a neuronal stem cell. Disposing the coating on the polymer complex can provide the polymer nanoparticle, for example, having a core-shell type structure.

The polymer nanoparticle can be prepared, for example, by contacting the crosslinked polymer complex with a cell to form a coating on the crosslinked polymer complex. For example, cells can be dispersed in an appropriate media, for example nuclease-free media. The hydrophobic polymer nanoparticle can be slowly added to the cell dispersion. Without wishing to be bound by theory, it is believed that the hydrophobic nature of the polymer nanoparticle will enable interaction with the native membrane of the cells. Such interactions can destabilize the cell membrane and provide the coating on the hydrophobic polymeric carrier. Accordingly, the process can be autonomous, wherein the cells and the polymer complexes are co-incubated. In an alternative process, the crosslinked polymer complex can be contacted with membrane lipids derived from a native cellular membrane.

The coated polymer nanoparticles can be separated from intact cells and/or cellular debris, for example using centrifugation. In an aspect, the coating can be uniformly distributed on the surface of the polymer complex. Optionally, the coated polymer nanoparticles can be further purified, for example using affinity column chromatography using antibodies against cell membrane-specific surface proteins.

The presence of the coating comprising the membrane derived from the cells can be confirmed, for example, using transmission electron microscopy (TEM).

Another aspect of the present disclosure relates to a method for delivering a cargo molecule (e.g., delivering the cargo intracellularly). The method comprises administering the polymer nanoparticle to a subject in need of therapy. Upon administration, the entrapped cargo can be released from the crosslinked polymer matrix at a target site. Any appropriate route of administration can be employed, for example parenteral, intravenous, subcutaneous, intramuscular, intraventricular, intracorporeal, intraperitoneal, rectal, or oral administration.

As used herein, the term “subject” refers to any animal (e.g., a mammal), including, for example, humans, non-human primates, rodents, and the like, which is to be the recipient of a particular treatment. The term “subject” may be used interchangeably with “patient”, particularly in reference to a human subject.

In an aspect, the polymer nanoparticle of the present disclosure can be provided (e.g., to a subject) in the form of a composition. The composition can comprise one or more pharmaceutically acceptable components, for example an excipient, a carrier, or a diluent. As used herein, the term “pharmaceutically acceptable” refers to a pharmaceutically acceptable material, composition, or vehicle, such as a liquid or solid filler, diluent, excipient, solvent or encapsulating material, involved in carrying or transporting the subject polymer nanoparticle. Each carrier must be “acceptable” in the sense of being compatible with the other ingredients of the formulation and not injurious to the subject. Exemplary pharmaceutical acceptable carriers can include, but are not limited to, sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; and other non-toxic compatible substances employed in pharmaceutical formulations. Wetting agents, emulsifiers and lubricants, such as sodium lauryl sulfate, magnesium stearate, and polyethylene oxide-polypropylene oxide copolymer as well as coloring agents, release agents, coating agents, sweetening, flavoring and perfuming agents, preservatives and antioxidants can also be present in the compositions.

The polymer nanoparticle or a composition comprising the polymer nanoparticle can be provided to a subject in an effective amount. As used herein, the term “effective amount” refers to an amount sufficient to elicit a desired biological response. As will be appreciated by those of skill in the art, the effective amount of a compound can vary depending on such factors as the desired biological endpoint, the pharmacokinetics of the compound, the disease being treated, the mode of administration, and the patient.

This disclosure is further illustrated by the following examples, which are non-limiting.

### EXAMPLES

The unique membrane activity of the polymer nanoparticles with a hydrophobic surface facilitates autonomous coating of the polymer nanoparticles with a variety of lipid compositions.

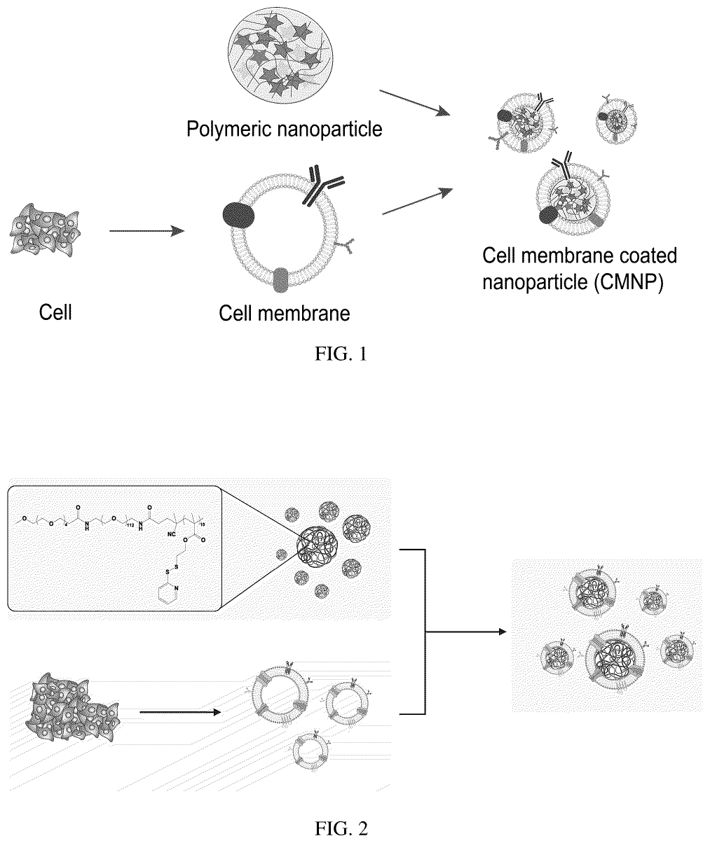

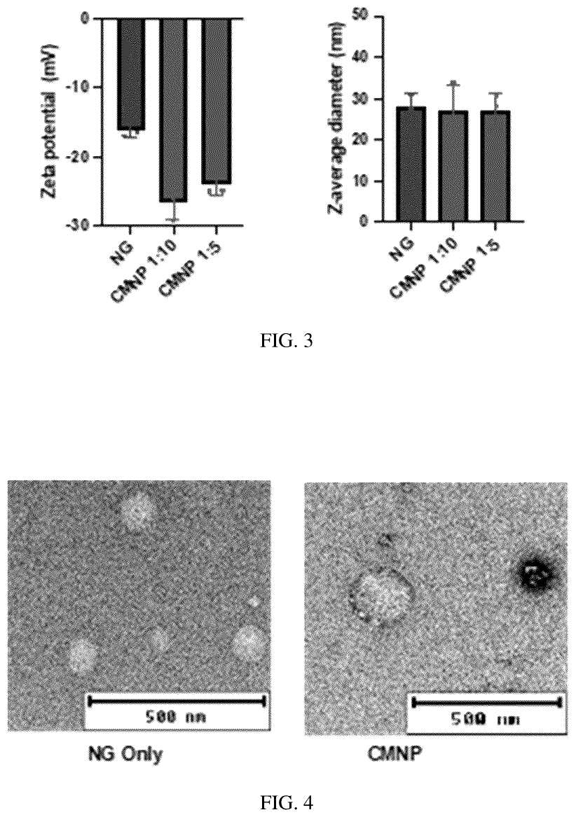

In a first example, crosslinked polymer nanoparticles with hydrophilic polyethyleneglycol (PEG) units and hydrophobic pyridyldisulfide (PDS) units with partially crosslinked format were coated with membranes derived from MDA-MB-468 cell lines (FIG. 2) and characterized using size and zeta potential measurements and transmission electron microscopy (TEM). A slight increase in the size and the change in the surface charge of the nanogels show that these PEG-PDS nanogels are indeed coated with cellular membrane materials, as shown in FIG. 3. FIG. 4 shows TEM images of the polymer nanoparticles alone (left) and after coating with the cellular membrane (right).

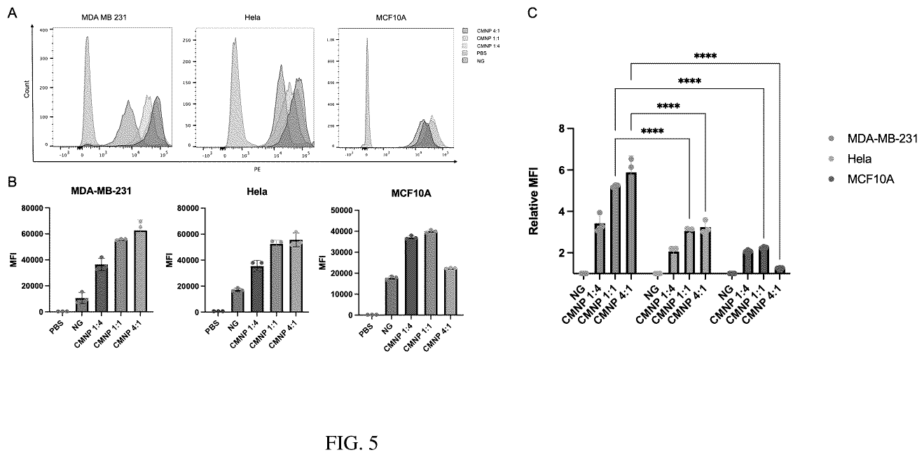

Different ratios of polymer to derived cell membrane have been investigated to optimize the coating of nanoparticles with cell membrane materials. Particles derived from MDA-MB-468 cells were then incubated with three different cell types i.e., MDA-MB-231, Hela and MCF10A cells and analyzed by flow cytometry, as shown in FIG. 5. As shown in FIG. 5, different polymer to cell membrane weight ratio formulations were tested, ranging from 1:4, to 1:1, to 4:1. Interestingly, MDA-MB-231 derived nanoparticles showed a much better uptake efficiency compared to the uncoated polymeric nanoparticles in all three cell lines. Also, between the cell lines, the cell membrane coated nanoparticles also exhibited cellular selectivity from confocal microscopy studies, where better uptake was observed with the host MDA-MB-468 cells, while almost no uptake was observed for MCF10A cells for the same incubation time.

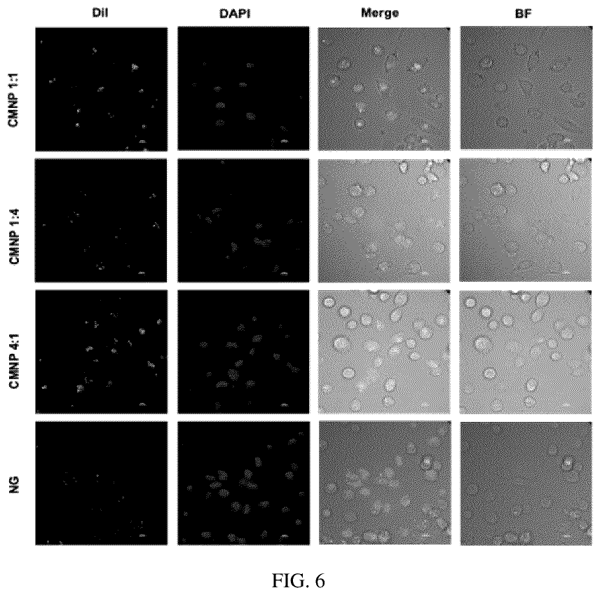

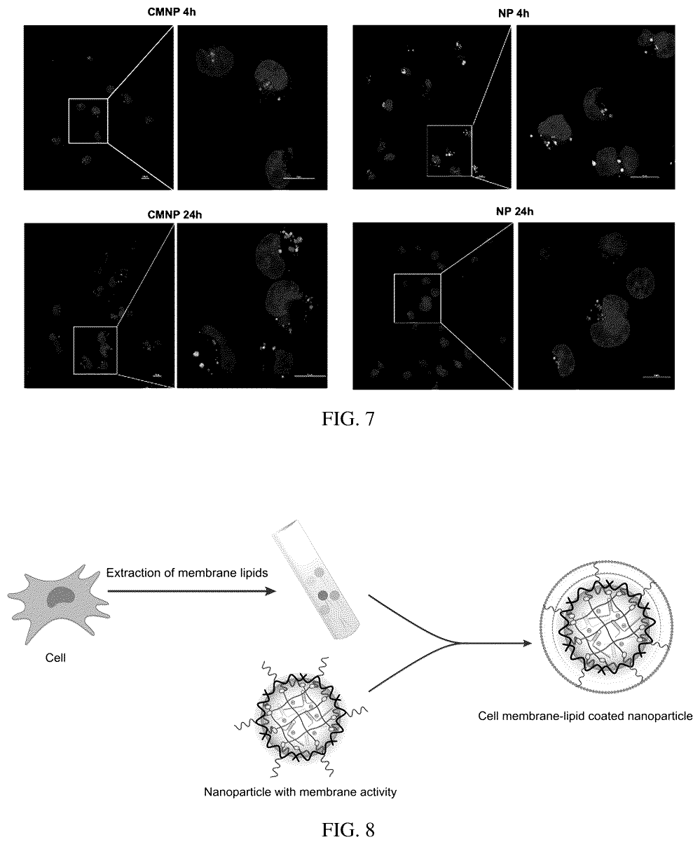

Confocal microscopy results showing enhanced cellular uptake of the polymer nanoparticles including the cellular coating are shown in FIG. 6. As shown in FIG. 6, increased uptake was observed in the case of the MDA-MB-231 cells, but limited uptake was observed for MCF10A cells. It was further observed that the coated polymer nanoparticles were taken up by the cells by membrane fusing, rather than an endosomal pathway (FIG. 7).

In addition to directly incorporating cellular membrane materials on the polymeric nanocarrier, pre-extracted lipid materials from cells were also coated on polymer nanoparticles. Here, it was hypothesized that such a bio-lipid coating of polymeric nanoparticles would offer facilitated cellular uptake and endosomal escape in a highly scalable format. A schematic illustration of this process is depicted in FIG. 8.

In a second example, polymer nanoparticles containing dodecyl functionality on the surface of the nanoparticle with a pyridinium disulfide-based interior that are partially crosslinked were treated with membrane lipids were derived from MDA-MB-231 cells using a modified Bligh and Dyer extraction method. Briefly, the cells were scraped from the culture plates and washed with DPBS to get rid of debris and media. Then the cell pellet was dissolved in 2:1 (v/v) methanol:dichloromethane (DCM) to dissolve the cell membranes. Finally, water and DCM were added, followed by vortexing and centrifugation to separate the organic and water layers. The bottom organic layer was aspirated out in a separate vial. The extraction process was repeated two more times to ensure the lipid purity. The amount of extracted lipid was measured colorimetrically using sulfo-phospho-vanillin (SPV) assay kit from Cell Biolabs.

The nanoparticles were formulated using a nano-precipitation technique. Briefly, dye labeled siRNA was mixed with polymer dissolved in 70:30 (v/v) ratio of acetone:water and shaken on an orbital shaker at 21° C. for 2 hours. Next, dithiothreitol (DTT) to crosslink the polymer complex. Finally, the isolated lipids were dissolved in water and the polymer complex was added dropwise and stirred at room temperature for 3 hours. Finally, the solutions were filtered through Amicon Ultra centrifugal filters MWCO 10 kDa to remove remaining organic solvents, purify, and concentrate the solutions.

To study the cellular uptake, cells were plated in a 96-well plate (2×10⁴ cells in each well) and incubated for 24 hours at 37° C. Cells were then transfected with Cy3-siRNA-loaded L-siP nanoassemblies and incubated for 4 hours. Finally, cells were trypsinized, pelleted by centrifugation, and washed two times with PBS followed by suspension in 500 μL of PBS. Flow cytometry was performed with this cell suspension in a BD LSRFortessa instrument (excitation wavelength, 562 nm; PE channel) to check the reduction in the GFP fluorescence intensity. FlowJo version 10 software was used to analyze data and obtain fluorescence intensities of the samples.

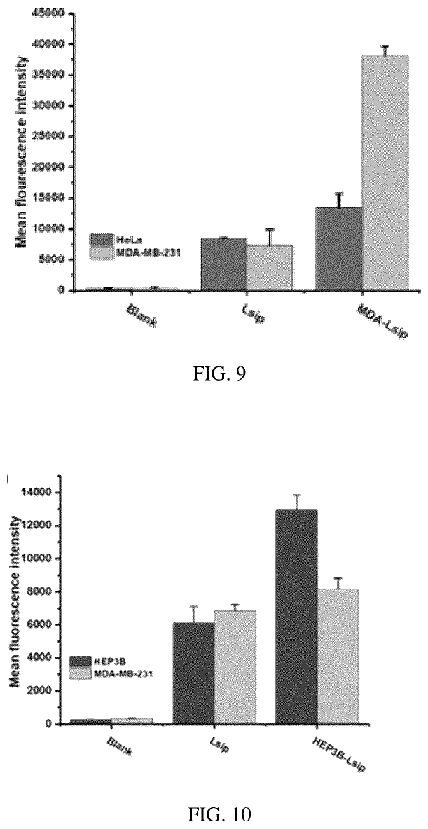

Two different cells (HeLa and MDA-MB-231) were subjected to the nanoparticles derived from MDA-MB-231 cells. Flow cytometry analysis of the uptake profiles in both cells substantiate the effect of possible homotypic selection to the parent cell, as shown in FIG. 9. Almost four times higher uptake of nanoparticles in MDA-MB-231 cells was observed compared to control HeLa cells.

HEP3B cells were also used as the membrane donor to test the generalizability of the present disclosute. The formed nanoparticles still show significantly more uptake tendency towards the donor cell, which again confirms the generalizability of the homotypic targeting concept using only cellular lipids (FIG. 10).

Experimental details follow.

Polymers used to prepare the polymer nanoparticles in the present examples were prepared, for example, according to procedures described in U.S. Publication No. 2019/0209698 and U.S. Publication No. 2020/0332047, both of which are incorporated by reference herein in the entirety.

Nanoparticle preparation: A weight ratio of 1:1 of tetrazine- and TCO-terminated PEG₅₀₀₀-PDS₁₀ were dissolved in dry DMSO to make 50 mg/mL of each polymer. The DiI was prepared at a concentration of 10 mg/mL in DMSO. 20 uL of each solution was then placed in an 8 mL vial along with the desired cargo prepared in DMSO solution. HPLC-grade water was then added to the DMSO solution, immediately followed by ultrasonication for 5 minutes. DMSO was purified by dialysis against DI waster using a mini dialysis kit (1 kDa cutoff) for 6 hours. DTT (7.7 uL; 5 mg/mL in water) was added to achieve a 20% crosslinking density. After 3 hours of crosslinking, nanoparticles were further purified using a mini dialysis kit (8 kDa cutoff) for 24 hours. The polymer nanoparticles were stored at 4 degrees for further use.

Cell membrane isolation: MDA-MB-468 cells were grown in 100 mm Cell Culture Dish (Corning) and harvested at a confluency of 70%. Cells were detached using a cell scraper. Cells were spun down at 300 ×g for 5 min, the cell pellet was resuspended in cell wash solution (30 mM Tris-HCL PH 7.5, 2.6% w/v sucrose and 4.1% w/v D-mannitol) and washed for three times. Then, the supernatant was discarded and resuspended the pellet in a permeabilization buffer (protease inhibitor, phosphatase inhibitor and 400 uM ethylene glycol-bis(β-aminoethyl ether)-N,N,N′,N′-tetraacetic acid and then mechanically lysed the solution using vortex/homogenizer at 4 degrees. Intracelluar contents were purified out by centrifuging the solution at 16,000 ×g for 15 min Finally, the supernatant was discarded and the solubilization buffer (200 uL EDTA in DNase free/RNase free water) was added. Centrifuged the solution at 16,000 ×g for 15 min at 4 degrees (similar results were also obtained with 150,000 ×g). The resulting membrane was stored at −20 degrees for further use.

Cell membrane-coated nanoparticles synthesis and characterization: The cell membrane solution and PEG₅₀₀₀-PDS₁₀ block copolymer were mixed at different volume ratios in a 2 mL glass vial, sonicated in the water bath for 5 minutes. Size and Zeta potential characterization was performed at a final concentration of polymer at 0.5 mg/mL in HPLC grade water in a semi-micro cuvette (Fisherbrand) and a folded capillary zeta cell (Malvern Panalytical) separately. In order to visualize the cellular uptake of the cell membrane-coated nanoparticles, 1×10⁴ cells were first plated onto a tissue culture-treated glass bottom dish and incubated at 37° C. overnight. Then, either bare nanoparticles or cell membrane-coated nanoparticles were added to the cultures at a final concentration of 20 μg/mL. After incubating at 37° C. for 30 min, wash the cell with PBS and add fresh new media to continue incubating for 4 hours. To prepare for visualization, the cells were washed with PBS and then stained with Hoechst 33342 (Invitrogen). Imaging was performed on a Nikon Ti2 stand with spinning disk confocal and 2 camera TIRF system.

Extraction of membrane lipids from live cells: Cells were plated in tissue culture dish until approximately 90% confluency was reached. Once they are confluent the cells were scraped off the plate and collected in a centrifuge tube followed by centrifugation to obtain a cell pellet. The collected cell pellet was washed for at least 2 times with cold PBS to remove any excess media. Number of obtained cells were calculated using an automated hemocytometer. Next, fixed amounts of three solvents, 1:2 chloroform-methanol mixture, chloroform, and 1.5 M NaCl were added to the obtained pellet with vortexing in between addition of different solvents. (Alharbi, H. M., et al. AAPS Open 4, 1-9 (2018)). Finally, the mixture was centrifuged at 1500 RPM at 4° C. to obtain a biphasic mixture. The bottom chloroform layer was aspirated and collected in a clean glass vial. The chloroform was evaporated by rotary evaporation followed by vacuum drying. The final obtained lipid amount was estimated gravimetrically, and the lipid was stored −20° C. until use.

Preparation of the membrane lipid coated nanoparticles: For preparation of the siRNA encapsulated nanoparticles a mixed solvent system (acetone/water=70:30) was used. At the beginning a fixed amount of Cy3-labeled negative control 1 siRNA (From Thermofisher) was dosed in acetone/water solvent mixture (2 μg) to get 50 μL solutions. In a separate vial 2 μg/μL solution of polymer was prepared in acetone/water solvent. 13.5 μL of the polymer solution (to achieve a N/P ratio of 15) was taken in a tube and diluted with additional acetone/water to obtain 50 μL solutions. After that, both the siRNA solution and the prepared polymer solutions have been mixed and shaken at 21° C. for approximately 2 hours followed by addition of 1 molar equivalent of dithiothreitol (DTT) (with respect to the PDS moiety in polymer) with additional 2 hours of shaking at 21° C. to prepare crosslinked nanoassemblies.

Meanwhile, mixed lipid solutions were prepared in 2 mL water containing 20 wt % of extracted membrane lipids/DOPE along with 10 wt % of DSPE-PEG2K and stirred to homogenize for 30 mins. Then 100 μL of nanoassembly solution was added to the lipid solution dropwise and stirred for 3 hours at 21° C. to facilitate the evaporation of the excess organic solvent. Finally, the solutions were filtered through Amicon Ultra centrifugal filters MWCO 10 kDa to remove remaining organic solvents and to concentrate the samples. The final volume was adjusted back to 100 μL with nuclease free water.

Uptake experiment: For uptake experiments, 1.5×10⁴ cells/well were plated in a 96 well tissue culture plate and incubated for 24 h at 37° C. in DMEM:F12 (1:1) supplemented with 10% FBS and 1% antibiotics (Purchased from Gibco). After that, the media was aspirated out from the plates followed by incubation of cells with prepared nanoparticles in serum free media for 4 h at 37° C. Finally, cells were trypsinized, pelted by centrifugation and resuspended in 35 μL of cold PBS. Flow cytometry analysis of these suspensions were performed in a BD LSRFortessa instrument (Excitation wavelength 555 nm; PE channel).

This disclosure further encompasses the following aspects.

Aspect 1: A polymer nanoparticle comprising a crosslinked polymer complex comprising a cargo encapsulated in a crosslinked polymer network; and a coating on the crosslinked polymer complex, wherein the coating is derived from a cellular membrane.

Aspect 2: The polymer nanoparticle of aspect 1, wherein the crosslinked polymer complex comprises: a crosslinked copolymer comprising repeating units of Formula (I) and (II)

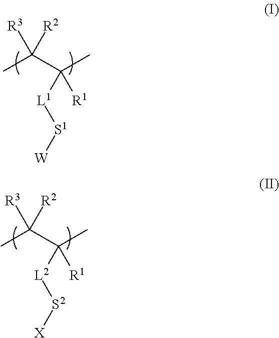

wherein in the foregoing Formulas R¹ is independently at each occurrence hydrogen, a C₁₋₁₂ alkyl group, or a halogen; R² and R³ are independently at each occurrence hydrogen, a C₁₋₆ alkyl group, a C₁₋₁₆ alkyloxy group, or halogen; L¹ and L² are independently at each occurrence a linking group; S¹ and S² are independently at each occurrence a single bond or a spacer group; W is a hydrophobic group or a hydrophilic group; and X is a group comprising a crosslinking moiety; and a cargo entrapped in the crosslinked copolymer.

Aspect 3: The polymer nanoparticle of aspect 1 or 2, wherein the crosslinked copolymer is a random or block copolymer.

Aspect 4: The polymer nanoparticle of any of aspects 1 to 3, wherein the crosslinked copolymer further comprises structural units of Formula (III)

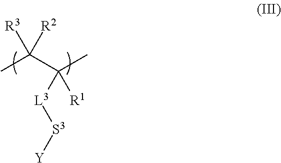

wherein R¹ is independently at each occurrence hydrogen, a C₁₋₁₂ alkyl group, or a halogen; R² and R³ are independently at each occurrence hydrogen, a C₁₋₆ alkyl group, a C₁₋₁₆ alkyloxy group, or halogen; L³ a linking group; S³ is a single bond or a spacer group; and Y is a non-crosslinking group.

Aspect 5: The polymer nanoparticle of any of aspects 1 to 4, wherein X comprises a crosslinked group.

Aspect 6: The polymer nanoparticle of any of aspects 1 to 5, wherein X comprises a group capable of forming a crosslinking bond.

Aspect 7: The polymer nanoparticle of any of aspects 1 to 6, wherein the cargo encapsulated in the crosslinked polymer matrix comprises a hydrophobic molecule, a nucleic acid, a protein, an antibody, or a combination thereof.

Aspect 8: The polymer nanoparticle of any of aspects 1 to 7, wherein the cargo encapsulated in the crosslinked polymer matrix comprises a nucleic acid, a protein, or a combination thereof.

Aspect 9: The polymer nanoparticle of any of aspects 1 to 8, wherein the cargo comprises a nucleic acid comprising single-stranded or double-stranded RNA or DNA, or a derivative or analog thereof.

Aspect 10: The polymer nanoparticle of any of aspects 1 to 9, wherein the cargo comprises a nucleic acid comprising dsRNA, siRNA, mRNA, ncRNA, microRNA, catalytic RNA, gRNA, DNAs, oligonucleotides, aptamers, genes, plasmids, or a derivative or analog thereof.

Aspect 11: The polymer nanoparticle of any of aspects 1 to 10, wherein the cargo comprises a nucleic acid comprising siRNA.

Aspect 12: The polymer nanoparticle of any of aspects 1 to 11, wherein the cargo comprises a protein comprising a CRISPR-associated protein, preferably Cas9.

Aspect 13: The polymer nanoparticle of any of aspects 1 to 12, wherein cargo comprises the nucleic acid and the protein.

Aspect 14: The polymer nanoparticle of any of aspects 2 to 13, wherein W comprises a C₁₋₃₀ linear or branched alkyl group.

Aspect 15: The polymer nanoparticle of any of aspects 4 to 14, wherein each of L¹, L², and L³ is independently an ester (—(C═O)O—) or an amide (—(C═O)NH—) linking group.

Aspect 16: The polymer nanoparticle of any of aspects 2 to 15, wherein X comprises a disulfide group.

Aspect 17: The polymer nanoparticle of any of aspects 2 to 16, wherein X comprises a group of the Formula

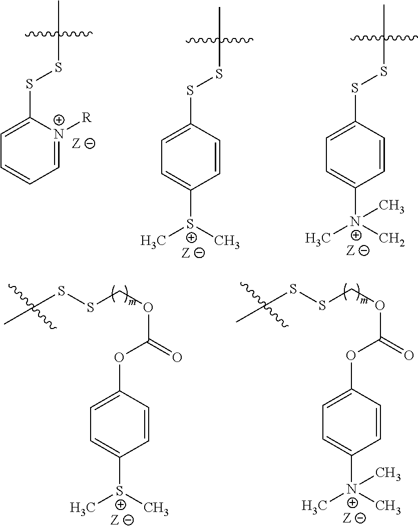

wherein R is a C₁₋₁₅ alkyl group, and Z is a counter ion.

Aspect 18: The polymer nanoparticle of any of aspects 1 to 17, wherein the crosslinked polymer network comprises a copolymer of Formula (IV)

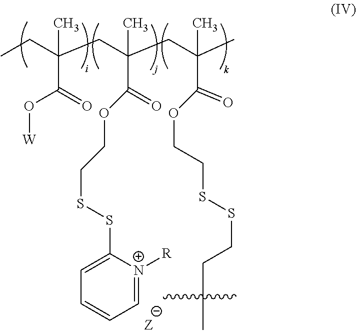

wherein W is a C₁₋₃₀ alkyl group, R is a C₁₋₁₅ alkyl group, Z is a counter ion, and i and j are independently at each occurrence an integer from 1 to 500, and k is an integer from 0 to 500.

Aspect 19: The polymer nanoparticle of any of aspects 1 to 18, wherein the polymer nanoparticle is crosslinked intermolecularly and intramolecularly.

Aspect 20: The polymer nanoparticle of any of aspects 1 to 19, wherein the polymer nanoparticle is adapted to de-crosslink partially or completely upon contact with a biological or chemical stimulus.

Aspect 21: The polymer nanoparticle of any of aspects 2 to 20, wherein X comprises a pH-sensitive functional group.

Aspect 22: The polymer nanoparticle of any of aspects 2 to 21, wherein X comprises a redox-sensitive functional group.

Aspect 23: The polymer nanoparticle of any of aspects 1 to 22, wherein the cell is a human cell, an animal cell, or a plant cell.

Aspect 24: The polymer nanoparticle of any of aspects 1 to 23, wherein the coating is derived from a blood cell, a tumor cell, a cancer cell, an immune cell, a stem cell, a neuronal cell, an epithelial cell, or an endothelial cell.

Aspect 25: A method of making the polymer nanoparticle of any of aspects 1 to 24, the method comprising: contacting an amphiphilic polymer and a cargo molecule to form a polymer complex; crosslinking the polymer to form a crosslinked polymer network entrapping the cargo molecule therein; and contacting the crosslinked polymer complex with a cell under conditions effective to provide the coating derived from a cellular membrane.

Aspect 26: A method for delivering a cargo, the method comprising: administering the polymer nanoparticle of any of aspects 1 to 24 to a subject in need of therapy.

The compositions, methods, and articles can alternatively comprise, consist of, or consist essentially of, any appropriate materials, steps, or components herein disclosed. The compositions, methods, and articles can additionally, or alternatively, be formulated so as to be devoid, or substantially free, of any materials (or species), steps, or components, that are otherwise not necessary to the achievement of the function or objectives of the compositions, methods, and articles.

All ranges disclosed herein are inclusive of the endpoints, and the endpoints are independently combinable with each other. “Combinations” is inclusive of blends, mixtures, alloys, reaction products, and the like. The terms “first,” “second,” and the like, do not denote any order, quantity, or importance, but rather are used to distinguish one element from another. The terms “a” and “an” and “the” do not denote a limitation of quantity, and are to be construed to cover both the singular and the plural, unless otherwise indicated herein or clearly contradicted by context. “Or” means “and/or” unless clearly stated otherwise. Reference throughout the specification to “an aspect” means that a particular element described in connection with the aspect is included in at least one aspect described herein, and may or may not be present in other aspects. The term “combination thereof” as used herein includes one or more of the listed elements, and is open, allowing the presence of one or more like elements not named. In addition, it is to be understood that the described elements may be combined in any suitable manner in the various aspects.

Unless specified to the contrary herein, all test standards are the most recent standard in effect as of the filing date of this application, or, if priority is claimed, the filing date of the earliest priority application in which the test standard appears.

Unless defined otherwise, technical and scientific terms used herein have the same meaning as is commonly understood by one of skill in the art to which this application belongs. All cited patents, patent applications, and other references are incorporated herein by reference in their entirety. However, if a term in the present application contradicts or conflicts with a term in the incorporated reference, the term from the present application takes precedence over the conflicting term from the incorporated reference.

Compounds are described using standard nomenclature. For example, any position not substituted by any indicated group is understood to have its valency filled by a bond as indicated, or a hydrogen atom. A dash (“−”) that is not between two letters or symbols is used to indicate a point of attachment for a substituent. For example, —CHO is attached through carbon of the carbonyl group.

As used herein, the term “hydrocarbyl”, whether used by itself, or as a prefix, suffix, or fragment of another term, refers to a residue that contains only carbon and hydrogen. The residue can be aliphatic or aromatic, straight-chain, cyclic, bicyclic, branched, saturated, or unsaturated. It can also contain combinations of aliphatic, aromatic, straight chain, cyclic, bicyclic, branched, saturated, and unsaturated hydrocarbon moieties. However, when the hydrocarbyl residue is described as substituted, it may, optionally, contain heteroatoms over and above the carbon and hydrogen members of the substituent residue. Thus, when specifically described as substituted, the hydrocarbyl residue can also contain one or more carbonyl groups, amino groups, hydroxyl groups, or the like, or it can contain heteroatoms within the backbone of the hydrocarbyl residue. The term “alkyl” means a branched or straight chain, saturated aliphatic hydrocarbon group, e.g., methyl, ethyl, n-propyl, i-propyl, n-butyl, s-butyl, t-butyl, n-pentyl, s-pentyl, and n- and s-hexyl. “Alkenyl” means a straight or branched chain, monovalent hydrocarbon group having at least one carbon-carbon double bond (e.g., ethenyl (—HC═CH₂)). “Alkoxy” means an alkyl group that is linked via an oxygen (i.e., alkyl-O—), for example methoxy, ethoxy, and sec-butyloxy groups. “Alkylene” means a straight or branched chain, saturated, divalent aliphatic hydrocarbon group (e.g., methylene (—CH₂—) or, propylene (—(CH₂)₃—)). “Cycloalkylene” means a divalent cyclic alkylene group, —CₙH₂ₙ₋ₓ, wherein x is the number of hydrogens replaced by cyclization(s). “Cycloalkenyl” means a monovalent group having one or more rings and one or more carbon-carbon double bonds in the ring, wherein all ring members are carbon (e.g., cyclopentyl and cyclohexyl). “Aryl” means an aromatic hydrocarbon group containing the specified number of carbon atoms, such as phenyl, tropone, indanyl, or naphthyl. “Arylene” means a divalent aryl group. “Alkylarylene” means an arylene group substituted with an alkyl group. “Arylalkylene” means an alkylene group substituted with an aryl group (e.g., benzyl). The prefix “halo” means a group or compound including one more of a fluoro, chloro, bromo, or iodo substituent. A combination of different halo atoms (e.g., bromo and fluoro), or only chloro atoms can be present. The prefix “hetero” means that the compound or group includes at least one ring member that is a heteroatom (e.g., 1, 2, or 3 heteroatom(s)), wherein the heteroatom(s) is each independently N, O, S, S¹, or P. “Substituted” means that the compound or group is substituted with at least one (e.g., 1, 2, 3, or 4) substituents that can each independently be a C₁₋₉ alkoxy, a C₁₋₉ haloalkoxy, a nitro (—NO₂), a cyano (—CN), a C₁₋₆ alkyl sulfonyl (—S(═O)₂-alkyl), a C₆₋₁₂ aryl sulfonyl (—S(═O)₂-aryl), a thiol (—SH), a thiocyano (—SCN), a tosyl (CH₃C₆H₄SO₂₋), a C₃₋₁₂ cycloalkyl, a C₂₋₁₂ alkenyl, a C₅₋₁₂ cycloalkenyl, a C₆₋₁₂ aryl, a C₇₋₁₃ arylalkylene, a C₄₋₁₂ heterocycloalkyl, and a C₃₋₁₂ heteroaryl instead of hydrogen, provided that the substituted atom's normal valence is not exceeded. The number of carbon atoms indicated in a group is exclusive of any substituents. For example —CH₂CH₂CN is a C₂ alkyl group substituted with a nitrile.

Unless substituents are otherwise specifically indicated, each of the foregoing groups can be unsubstituted or substituted, provided that the substitution does not significantly adversely affect synthesis, stability, or use of the compound. “Substituted” means that the compound, group, or atom is substituted with at least one (e.g., 1, 2, 3, or 4) substituents instead of hydrogen, where each substituent is independently nitro (—NO₂), cyano (—CN), hydroxy (—OH), halogen, thiol (—SH), thiocyano (—SCN), C₁₋₆ alkyl, C₂₋₆ alkenyl, C₂₋₆ alkynyl, C₁₋₆ haloalkyl, C₁₋₉ alkoxy, C₁₋₆ haloalkoxy, C₃₋₁₂ cycloalkyl, C₅₋₁₈ cycloalkenyl, C₆₋₁₂ aryl, C₇₋₁₃ arylalkylene (e.g., benzyl), C₇₋₁₂ alkylarylene (e.g, toluyl), C₄₋₁₂ heterocycloalkyl, C₃₋₁₂ heteroaryl, C₁₋₆ alkyl sulfonyl (—S(═O)₂-alkyl), C₆₋₁₂ arylsulfonyl (—S(═O)₂-aryl), or tosyl (CH₃C₆H₄SO₂₋), provided that the substituted atom's normal valence is not exceeded, and that the substitution does not significantly adversely affect the manufacture, stability, or desired property of the compound. When a compound is substituted, the indicated number of carbon atoms is the total number of carbon atoms in the compound or group, including those of any substituents.

While particular aspects have been described, alternatives, modifications, variations, improvements, and substantial equivalents that are or may be presently unforeseen may arise to applicants or others skilled in the art. Accordingly, the appended claims as filed and as they may be amended are intended to embrace all such alternatives, modifications variations, improvements, and substantial equivalents.

What is claimed is:

1. A polymer nanoparticle comprising
a crosslinked polymer complex comprising a cargo encapsulated in a crosslinked polymer network; and
a coating on the crosslinked polymer complex, wherein the coating is derived from a cellular membrane.

2. The polymer nanoparticle of claim 1, wherein the crosslinked polymer complex comprises:
a crosslinked copolymer comprising repeating units of Formula (I) and (II) 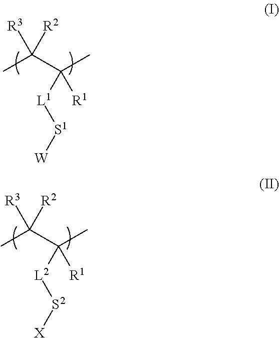
wherein in the foregoing Formulas
R¹ is independently at each occurrence hydrogen, a C₁₋₁₂ alkyl group, or a halogen;
R² and R³ are independently at each occurrence hydrogen, a C₁₋₆ alkyl group, a C₁₋₁₆ alkyloxy group, or halogen;
L¹ and L² are independently at each occurrence a linking group;
S¹ and S² are independently at each occurrence a single bond or a spacer group;
W is a hydrophobic group or a hydrophilic group; and
X is a group comprising a crosslinking moiety; and
a cargo entrapped in the crosslinked copolymer.

3. The polymer nanoparticle of claim 1, wherein the crosslinked copolymer further comprises structural units of Formula (III) 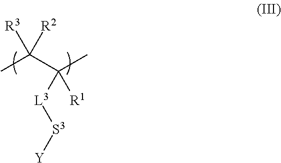
wherein
R¹ is independently at each occurrence hydrogen, a C₁₋₁₂ alkyl group, or a halogen;
R² and R³ are independently at each occurrence hydrogen, a C₁₋₆ alkyl group, a C₁₋₁₆ alkyloxy group, or halogen;
L³ a linking group;
S³ is a single bond or a spacer group; and
Y is a non-crosslinking group.

4. The polymer nanoparticle of claim 1, wherein X comprises a crosslinked group or a group capable of forming a crosslinking bond.

5. The polymer nanoparticle of claim 1, wherein the cargo encapsulated in the crosslinked polymer matrix comprises a hydrophobic molecule, a nucleic acid, a protein, an antibody, or a combination thereof.

6. The polymer nanoparticle of claim 1, wherein the cargo comprises a nucleic acid comprising single-stranded or double-stranded RNA or DNA, or a derivative or analog thereof; or
dsRNA, siRNA, mRNA, ncRNA, microRNA, catalytic RNA, gRNA, DNAs,
oligonucleotides, aptamers, genes, plasmids, or a derivative or analog thereof.

7. The polymer nanoparticle of claim 1, wherein the cargo comprises a nucleic acid comprising siRNA.

8. The polymer nanoparticle of claim 1, wherein the cargo comprises a protein comprising a CRISPR-associated protein.

9. The polymer nanoparticle of claim 1, wherein cargo comprises the nucleic acid and the protein.

10. The polymer nanoparticle of claim 2, wherein W comprises a C₁₋₃₀ linear or branched alkyl group.

11. The polymer nanoparticle of claim 3, wherein each of L¹, L², and L³ is independently an ester (—(C═O)O—) or an amide (—(C═O)NH—) linking group.

12. The polymer nanoparticle of claim 2, wherein X comprises a group of the Formula 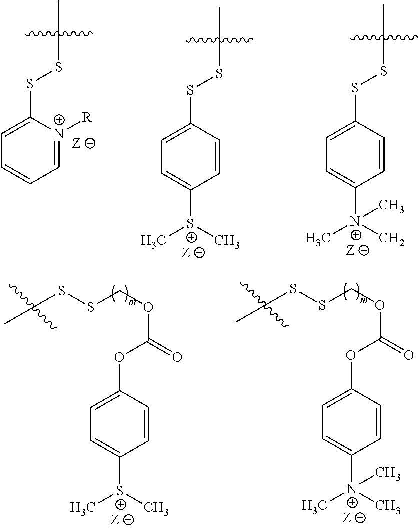
wherein
R is a C₁₋₁₅ alkyl group, and
Z is a counter ion.

13. The polymer nanoparticle of claim 1, wherein the crosslinked polymer network comprises a copolymer of Formula (IV) 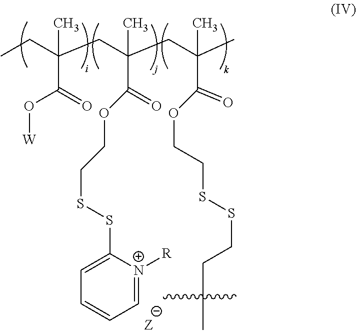
wherein
W is a C₁₋₃₀ alkyl group,
R is a C₁₋₁₅ alkyl group,
Z is a counter ion, and
i and j are independently at each occurrence an integer from 1 to 500, and
k is an integer from 0 to 500.

14. The polymer nanoparticle of claim 1, wherein the polymer nanoparticle is crosslinked intermolecularly and intramolecularly.

15. The polymer nanoparticle of claim 1, wherein the polymer nanoparticle is adapted to de-crosslink partially or completely upon contact with a biological or chemical stimulus.

16. The polymer nanoparticle of claim 2, wherein X comprises a pH-sensitive functional group or a redox-sensitive functional group.

17. The polymer nanoparticle of claim 1, wherein the coating is derived from a human cell, an animal cell, or a plant cell.

18. The polymer nanoparticle of claim 1, wherein the coating is derived from a blood cell, a tumor cell, a cancer cell, an immune cell, a stem cell, a neuronal cell, an epithelial cell, or an endothelial cell.

19. A method of making the polymer nanoparticle of claim 1, the method comprising:
contacting an amphiphilic polymer and a cargo molecule to form a polymer complex;
crosslinking the polymer to form a crosslinked polymer network entrapping the cargo molecule therein; and
contacting the crosslinked polymer complex with a cell under conditions effective to provide the coating derived from a cellular membrane.

20. A method for delivering a cargo, the method comprising:
administering the polymer nanoparticle of claim 1 to a subject in need of therapy.
